(19) United States
(12) Patent Application Publication
(10) Pub. No.: US 2014/0081197 A1
(43) Pub. Date: Mar. 20, 2014

(54) SURGICAL PRESSURIZATION DEVICE AND METHODS OF USING THE SAME

(71) Applicant: Robert S. Farivar, North Liberty, IA (US)

(72) Inventor: Robert S. Farivar, North Liberty, IA (US)

(21) Appl. No.: 13/991,016

(22) PCT Filed: Dec. 1, 2011
(86) PCT No.: PCT/US11/62880

§ 371 Date: Nov. 18, 2013

Related U.S. Application Data
(60) Provisional application No. 61/419,577, filed on Dec. 3, 2010.

(51) Int. Cl. A61M 1/00

(52) U.S. Cl. CPC A61M 1/0058
USPC 604/24

(57) ABSTRACT

A system for selectively pressurizing ventricles, atria, and the aorta of a subject is disclosed. The surgical pressunzation system has a gas container, a fluid container, and a surgical pressurization device. The gas container and the fluid container are in fluid communication with the surgical pressurization device. The gas container contains a pressurized gas, and the fluid container contains an irrigation fluid. The surgical pressurization device has a housing and a delivery conduit in fluid communication with the housing. The delivery conduit selectively delivers both the pressurized gas and the irrigation fluid into the ventricles, atria, and the aorta of the subject. A controller is used to control the flow of the pressurized gas and the irrigation fluid to the delivery conduit.

### CROSS-REFERENCE TO RELATED APPLICATIONS

This patent application claims priority to U.S. Provisional Patent Application Ser. No. 61/419,577, entitled “SURGICAL PRESSURIZATION DEVICE AND METHODS OF USING SAME” and filed on Dec. 3, 2010, the entirety of which is incorporated herein by reference.

### FIELD

This invention relates to devices, systems, and methods for pressurizing at least one of a ventricle, an atrium, and the aorta of a subject. More particularly, the invention relates to devices, systems, and methods for pressurizing the heart of a subject by delivering both a pressurized gas and an irrigation fluid into at least one of a left ventricle, a right ventricle, a left atrium, a right atrium, and the aorta of the subject.

### BACKGROUND

During performance of an open-heart surgery on a subject, it is necessary to eliminate or displace air within the subject's blood and to selectively pressurize the heart or aorta of the subject to detect the presence of leaks across a particular heart valve. Conventionally, air is displaced by flooding the surgical field with carbon dioxide gas in a process that relies on passive flow of the carbon dioxide gas into selected heart chambers (atria and ventricles) or the aorta. However, the carbon dioxide gas frequently fails to adequately flow into the heart or the aorta and, consequently, fails to displace the air within the subject's blood. Additionally, flooding of the surgical field in this manner can promote clot formation and lead to unintended entry of the carbon dioxide gas into the venting system of a cardiopulmonary bypass circuit, necessitating additional maneuvers to maintain a physiologic gas range.

Conventionally, selected chambers of the subject's heart and/or the aorta of the subject are pressurized using a bulb irrigator, which is configured to perform two functions: (1) ejecting fluid into the heart and/or the aorta; and (2) sucking fluid out of the heart and/or the aorta. Typically, the bulb irrigator is used to eject fluid into the selected heart chamber(s) and/or the aorta, thereby sealing the chamber(s) and/or aorta with the fluid. By monitoring the regurgitation of flow, a medical practitioner can then determine whether one or more heart valves contain leaks. Then, suction may be applied to the selected heart chamber(s) and/or the aorta, thereby collapsing the selected chamber(s) and/or the aorta and withdrawing unwanted fluid. However, the introduction of the fluid into the heart chamber(s) and/or the aorta may lead to additional air within the subject's blood, thereby increasing the likelihood that the subject may experience a stroke or heart attack due to air emboli. Additionally, the inadvertent passage of air through the coronary arteries may prolong the length of time spent accomplishing separation from bypass, even without an ischemic event. The separate flooding of the surgical field with carbon dioxide gas often fails to sufficiently displace the air within the subject's blood.

Accordingly, there is a need in the pertinent art for devices, systems, and methods for safely and efficiently eliminating air from the blood of a subject and testing one or more heart valves of a subject. More specifically, there is a need in the pertinent art for a single device or system for simultaneously or sequentially delivering both carbon dioxide gas and an irrigation fluid into at least one of a ventricle, an atrium, and an aorta of a subject.

### SUMMARY

Described herein is a surgical pressurization device for pressurizing at least one of a ventricle, an atrium, and an aorta of a subject. In one aspect, the surgical pressurization device has a housing and a delivery conduit. The housing defines a receiving chamber, which has a gas inlet for receiving a pressurized gas and a fluid inlet for receiving an irrigation fluid. The delivery conduit is in fluid communication with the receiving chamber of the housing. The delivery conduit has an outer tip portion having an opening. The delivery conduit is configured to deliver both the pressurized gas and the irrigation fluid through the opening of the outer tip portion and into the ventricles, atria, or the aorta of the subject. The surgical pressurization device can be incorporated into a surgical pressurization system having a gas container, a fluid container, and a suction source. Methods of using the disclosed devices and systems are also described.

### BRIEF DESCRIPTION OF THE FIGURES

These and other features of the preferred embodiments of the invention will become more apparent in the detailed description in which reference is made to the appended drawings wherein:

FIG. 1 is a schematic depiction of an exemplary surgical pressurization system, as described herein.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a delivery conduit” can include two or more such delivery conduits unless the context indicates otherwise.

As used herein, the term “cardiac region” means a chamber or other portion of a subject's body positioned adjacent to and in communication with one or more heart valves, including the mitral valve, the tricuspid valve, and the aortic valve. Thus, for example, the term “cardiac region” can include the left ventricle, the right ventricle, the left atrium, the right atrium, and the aorta.

As used herein, the terms “optional” or “optionally” mean that the subsequently described event or circumstance may or may not occur, and that the description includes instances where said event or circumstance occurs and instances where it does not.

As used herein, “simultaneous” and “simultaneously” refer to any two or more actions that exist or occur at substantially the same time. However, as used herein, two actions can occur “simultaneously” even though they are not exactly coincident and are merely overlapping. For example, two actions can occur “simultaneously” even though (a) the first action starts before the second action starts, (b) the second action continues after the first action ends, or (c) the first action starts before the second action starts and the first action continues after the second action ends.

It is contemplated that the disclosed devices and systems can comprise elements of the devices and systems described in U.S. Pat. Nos. 5,484,402, 6,213,970, 6,645,197, and 6,652,488, the disclosures of which are incorporated by reference herein in their entireties.

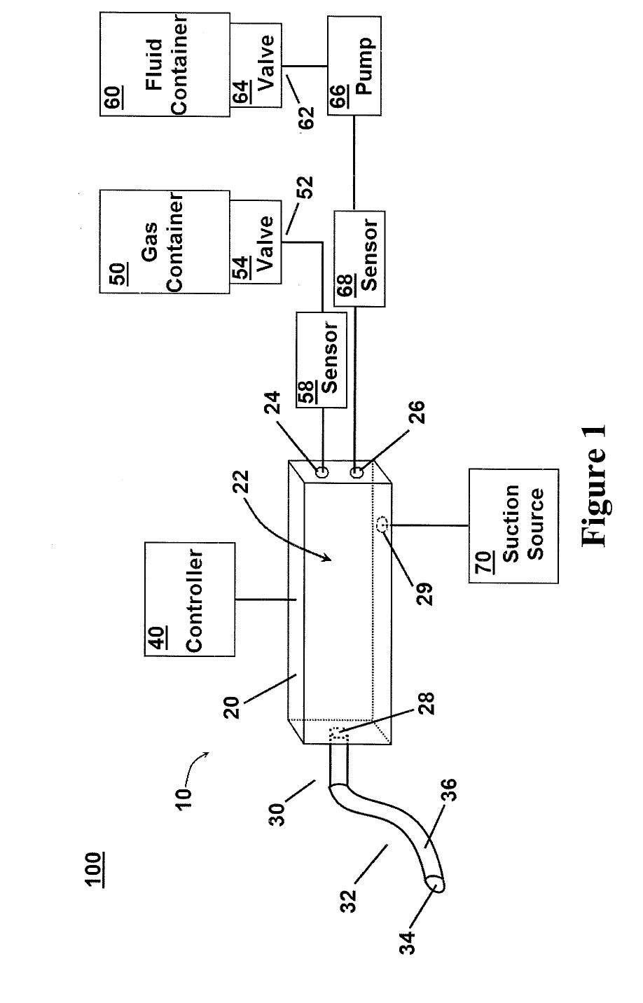

In one embodiment, and with reference to FIG. 1, the invention relates to a surgical pressurization device for pressurizing at least one cardiac region of a subject. It is contemplated that each cardiac region of the at least one cardiac region can be selected from the group consisting of a ventricle, an atrium, and the aorta of the subject. More particularly, it is contemplated that each cardiac region can be selected from the group consisting of the left atrium, the right atrium, the left ventricle, the right ventricle, and the aorta of the subject. In one aspect, it is contemplated that the surgical pressurization device can be used to pressurize a single selected cardiac region. In another aspect, the surgical pressurization device can be used to pressurize multiple selected cardiac regions. In various aspects, the surgical pressurization device can comprise a housing and a delivery conduit.

In one aspect, and as shown in FIG. 1, the housing 20 of the surgical pressurization device 10 can define a receiving chamber 22. In another aspect, the receiving chamber 22 can have a gas inlet 24 and a fluid inlet 26. Although the gas inlet 24 and the fluid inlet 26 are shown in FIG. 1 as distinct inlets, it is contemplated that the gas inlet and the fluid inlet can be combined into a single inlet. In an additional aspect, the gas inlet 24 can be configured to receive a pressurized gas, and the fluid inlet 26 can be configured to receive an irrigation fluid. In exemplary aspects, the pressurized gas can comprise pressurized carbon dioxide and the irrigation fluid can comprise saline solution. It is contemplated that carbon dioxide gas can diffuse rapidly in the blood of subjects and displace undesired air, thereby reducing (1) the time spent de-airing a subject undergoing a surgical procedure and (2) the potential risk of strokes and heart attacks in subjects undergoing surgical procedures. It is further contemplated that the saline solution can be either cold or warm saline solution. In a further aspect, it is contemplated that at least a portion of the housing 20 can be shaped for positioning within a hand of a user. For example, it is contemplated that the housing can comprise a handle that is shaped to complement the shape of a hand of a user.

In an additional aspect, and with reference to FIG. 1, the delivery conduit 30 of the surgical pressurization device 10 can be in fluid communication with the receiving chamber 22 of the housing 20. In this aspect, the housing 20 can have a port 28 at the interface between the delivery conduit 30 and the receiving chamber 22. Optionally, the pressurized gas and the irrigation fluid can be maintained separately within a portion of the housing 20, provided the pressurized gas and the irrigation fluid mix together prior to exiting the housing through port 28 and entering delivery conduit 30. In a further aspect, the delivery conduit 30 can have an outer tip portion 32. In this aspect, the outer tip portion 32 of the delivery conduit 30 can define an opening 34. Optionally, it is contemplated that the outer tip portion 32 can have a soft outer surface 36. It is further contemplated that the soft outer surface of the outer tip portion 32 can reduce the risk of puncturing or otherwise damaging a heart valve, ventricle, atrium, aorta or other portion of the subject's body. In one exemplary aspect, the outer tip portion 32 can comprise rubber. In another exemplary aspect, it is contemplated that the outer tip portion 32 can be configured to receive, and promote removal of, blood and/or air from one or more cardiac regions of a subject. In another aspect, the delivery conduit 30 can be configured to deliver both the pressurized gas and the irrigation fluid through the opening 34 of the outer tip portion 32 and into the selected cardiac regions of the subject. In this aspect, it is contemplated that the delivery conduit 30 can be configured to substantially simultaneously or contemporaneously deliver both the pressurized gas and the irrigation fluid into the selected cardiac regions of the subject.

As shown in FIG. 1, in a further aspect, the receiving chamber 22 of the housing 20 can comprise a waste outlet 29. In this aspect, the waste outlet 29 of the receiving chamber 22 can be selectively connectable in fluid communication with a suction source 70 for providing suction during activation. In an additional aspect, it is contemplated that, when the waste outlet 29 is connected in fluid communication with the activated suction source 70, undesired blood, fluid, and gas within the heart of the subject can be selectively removed through the opening 34 of the outer tip portion 32 of the delivery conduit 30 and transported through the receiving chamber 22 and the waste outlet 29 of the housing 20 before reaching the suction source. Thus, it is contemplated that the delivery conduit 30 can, in effect, selectively serve as either a suction head or a delivery device for the pressurized gas and the irrigation fluid.

In another aspect, as depicted in FIG. 1, the surgical pressurization device 10 can comprise a controller 40. In one aspect, the controller 40 can comprise a user interface. In this aspect, the user interface of the controller 40 can comprise one or more buttons that are configured to receive one or more inputs from a user. It is contemplated that the user can enter an input into the user interface by selectively depressing the one or more buttons. Additionally, the user interface can comprise one or more switches that are configured to receive one or more inputs from a user. It is contemplated that the user can enter an input into the user interface by selectively adjusting the position of the switches. In additional aspects, the user interface can receive inputs associated with one or more of the following functions: activation of the surgical pressurization device; creating flow of at least one of the pressurized gas and the irrigation fluid into the delivery conduit; and activation of a suction source. Thus, in response to the one or more user inputs, the controller 40 can selectively turn on the surgical pressurization device 10, direct (initiate, cease, or adjust) flow of at least one of the pressurized gas and the irrigation fluid into the delivery conduit 30, and/or activate the suction source 70. Optionally, in one aspect, it is contemplated that the controller 40 can be mounted thereon or coupled thereto the housing 20.

In exemplary aspects, as shown in FIG. 1, the surgical pressurization device 10 as described herein can function within a surgical pressurization system 100 for pressurizing at least one cardiac region of the subject. In one aspect, the surgical pressurization system 100 can comprise a gas container 50 for receiving the pressurized gas. In this aspect, the gas container 50 can have an outlet 52. It is contemplated that the gas inlet 24 of the receiving chamber 22 can be in fluid communication with the outlet 52 of the gas container 50. In one aspect, the gas inlet 24 of the receiving chamber 22 can be in fluid communication with the outlet 52 of the gas container 50 through conventional tubing. In an additional aspect, the gas container 50 can comprise a valve 54 for controlling the flow of the pressurized gas through the outlet 52 of the gas container 50. In this aspect, although not shown in FIG. 1, it is contemplated that the controller 40 of the surgical pressurization device 10 can be connectable in communication with the valve 54 of the gas container 50 through an electrical communication link, which can be any conventional means for electrical communication, including, for example and without limitation, conventional wires and conventional wireless transmission mechanisms, including, for example and without limitation, radio frequency (RF) communication mechanisms and infrared (IR) communication mechanisms. It is further contemplated that, in response to one or more user inputs, the controller 40 can be configured to control the valve 54 of the gas container 50 by opening and closing the valve to selectively control the flow of the pressurized gas through the outlet 52 of the gas container and to the gas inlet 24 of the receiving chamber 22.

As depicted in FIG. 1, in another aspect, the surgical pressurization system 100 can comprise a fluid container 60. In this aspect, the fluid container 60 can have an outlet 62. It is contemplated that the fluid inlet 26 of the receiving chamber 22 can be in fluid communication with the outlet 62 of the fluid container 60. In one aspect, the fluid inlet 26 of the receiving chamber 22 can be in fluid communication with the outlet 62 of the fluid container 60 through conventional tubing. In an additional aspect, the fluid container 60 can comprise a valve 64 for controlling the flow of the irrigation fluid through the outlet 62 of the fluid container 60. In this aspect, although not shown in FIG. 1, it is contemplated that the controller 40 of the surgical pressurization device 10 can be connectable in communication with the valve 64 of the fluid container 60 through an electrical communication link, which can be any conventional means for electrical communication, including, for example and without limitation, conventional wires and conventional wireless transmission mechanisms, including, for example and without limitation, radio frequency (RF) communication mechanisms and infrared (IR) communication mechanisms. It is further contemplated that, in response to one or more user inputs, the controller 40 can be configured to control the valve 64 of the fluid container 60 by opening and closing the valve to selectively control the flow of the irrigation fluid through the outlet 62 of the fluid container and to the fluid inlet 26 of the receiving chamber 22.

In one exemplary aspect, when the user input corresponds to a desired flow rate of the pressurized gas into the gas inlet 24, the controller 40 can be configured to selectively open or close the valve 54 of the gas container 50 to achieve the desired flow rate. In this aspect, it is contemplated that the surgical pressurization system 100 can comprise a flow sensor 58 positioned therebetween, and in fluid communication with, the outlet 52 of the gas container 50 and the gas inlet 24 of the receiving chamber 22. Although not shown in FIG. 1, it is further contemplated that the flow sensor 58 can be positioned in electrical communication with the controller 40. It is still further contemplated that the flow sensor 58 can be configured to generate a flow signal indicative of the flow rate of the pressurized gas into the gas inlet 24. It is still further contemplated that the flow sensor 58 can be configured to transmit the flow signal to the controller 40.

In a further aspect, and with reference to FIG. 1, the surgical pressurization system 100 can comprise a suction source 70. In this aspect, the suction source 70 can be selectively activated to provide suction. It is contemplated that the suction source 70 can be a conventional wall suction source. Alternatively, the suction source 70 can be a conventional portable suction source. In an additional aspect, the receiving chamber 22 of the housing 20 can comprise a waste outlet 29 selectively connectable in fluid communication with the suction source 70 such that vacuum suction can be applied through the waste outlet. In this aspect, it is contemplated that, when the waste outlet 29 is connected in fluid communication with the activated suction source 70, undesired blood, fluid, and gas within the heart of the subject can be removed through the opening 34 of the outer tip portion 32 of the delivery conduit 30 and transported through the receiving chamber 22, out the waste outlet 29, and toward the suction source 70. It is further contemplated that the undesired blood, fluid, and gas can be collected using conventional methods after exiting the waste outlet 29. It is further contemplated that, when the waste outlet 29 is connected in fluid communication with the activated suction source 70, the selected cardiac regions of the subject can be collapsed in a desired manner. In one aspect, the waste outlet 29 can be in fluid communication with the suction source 70 through conventional tubing that is configured to form a fluid-tight connection with the waste outlet. In this aspect, it is contemplated that the tubing can be coupled to the waste outlet 29 using any conventional means for forming a fluid-tight connection. For example, in one aspect, the waste outlet 29 can be configured to receive the tubing in a fluid-tight manner. In another exemplary aspect, a portion of the waste outlet 29 can protrude from the housing 20, and the tubing can be configured to receive the protruding portion of the waste outlet in a fluid-tight manner.

Optionally, although not shown in FIG. 1, the controller 40 of the surgical pressurization device 10 can be connectable in communication with the suction source 70 through an electrical communication link, which can be any conventional means for electrical communication, including, for example and without limitation, conventional wires and conventional wireless transmission mechanisms, including, for example and without limitation, radio frequency (RF) communication mechanisms and infrared (IR) communication mechanisms. It is contemplated that, in response to one or more user inputs, the controller 40 can be configured to control activation of the suction source 70.

In still a further aspect, and as depicted in FIG. 1, the surgical pressurization system 100 can comprise a pump 66 in fluid communication with the outlet 62 of the fluid container 60. In this aspect, the pump 66 can have a motor configured to pump irrigation fluid into the fluid inlet 26 of the receiving chamber 22. In one aspect, the pump 66 can be connected between the outlet 62 of the fluid container 60 and the fluid inlet 26 of the receiving chamber 22 using conventional tubing. It is contemplated that the controller 40 of the surgical pressurization device 10 can be connectable in fluid communication with the pump 66 through an electrical communication link, which can be any conventional means for electrical communication, including, for example and without limitation, conventional wires and conventional wireless transmission mechanisms, including, for example and without limitation, radio frequency (RF) communication mechanisms and infrared (IR) communication mechanisms. It is further contemplated that, in response to one or more user inputs, the controller 40 can be configured to adjust the motor of the pump 66 to selectively control the flow of the irrigation fluid into the fluid inlet 26 of the receiving chamber 22. In one exemplary aspect, when the user input corresponds to a desired flow rate of the irrigation fluid into the fluid inlet 26, the controller 40 can be configured to adjust the motor of the pump 66 to achieve the desired flow rate. Additionally, it is contemplated that the surgical pressurization system 100 can comprise a flow sensor 68 positioned therebetween, and in fluid communication with, the outlet 62 of the fluid container 60 and the fluid inlet 26 of the receiving chamber 22. Although not shown in FIG. 1, it is further contemplated that the flow sensor 68 can be positioned in electrical communication with the controller 40. It is still further contemplated that the flow sensor 68 can be configured to generate a flow signal indicative of the flow rate of the irrigation fluid into the fluid inlet 26. It is still further contemplated that the flow sensor 68 can be configured to transmit the flow signal to the controller 40.

In some aspects, the controller 40 can comprise a processor. In these aspects, the processor can be programmed to operate in accordance with at least one of software, firmware, and field-programmable gate array (FPGA) code. It is contemplated that the controller 40 can comprise a memory that is configured to store the software, firmware, and FPGA code that control the operation of the processor. Alternatively, the controller 40 can be in communication with an external computer that stores the software, firmware and FPGA code. In one aspect, at least one of the software, firmware, and FPGA code can instruct the controller 40 to determine whether desired flow rates of the irrigation fluid and/or the pressurized gas are achieved, as disclosed herein. In still a further aspect, the controller 40 can be in communication with a conventional display. In this aspect, during use of the disclosed devices and systems, the controller 40 can be configured to display one or more outputs, such as, for example and without limitation, monitored flow rates of the irrigation fluid and/or the pressurized gas.

In use, the disclosed devices and systems can be incorporated into methods for pressurizing at least one cardiac region of a subject. In one aspect, the methods can comprise establishing fluid communication between the outlet of the gas container and the gas inlet of the receiving chamber of the surgical pressurization device. In another aspect, the methods can comprise establishing fluid communication between the outlet of the fluid container and the fluid inlet of the receiving chamber of the surgical pressurization device. In an additional aspect, the methods can comprise positioning the outer tip portion of the delivery conduit of the surgical pressurization device through a heart valve and into a first selected cardiac region of the subject. In a further aspect, the methods can comprise selectively delivering at least one of the pressurized gas and irrigation fluid into the first selected cardiac region of the subject. It is contemplated that the step of selectively delivering at least one of the pressurized gas and the irrigation fluid can comprise substantially simultaneously or contemporaneously delivering both the pressurized gas and the irrigation fluid into the first selected cardiac region of the subject. Optionally, in one exemplary aspect, the methods can further comprise positioning the outer tip portion of the delivery conduit of the surgical pressurization device through a heart valve and into a second selected cardiac region of the subject and selectively delivering at least one of the pressurized gas and irrigation fluid into the second selected cardiac region of the subject. In this aspect, it is contemplated that the same-heart valve can be used to access both the first and second selected cardiac regions. Alternatively, it is contemplated that a first heart valve can be used to access the first selected cardiac region and a second heart valve can be used to access the second selected cardiac region.

In these aspects, it is contemplated that the heart valve can be selected from the group consisting of a mitral valve, a tricuspid valve, and an aortic valve. For example, and without limitation, it is contemplated that the outer tip portion can be positioned through a mitral valve to pressurize the left ventricle of the subject during an open heart procedure. It is contemplated that the surgical pressurization devices and systems can be used to directly and rapidly pressurize the selected cardiac regions of the subject to thereby allow for detection of leaks across the heart valve through which the delivery conduit is inserted. More particularly, it is contemplated that the surgical pressurization devices and systems can be used to fill the selected cardiac regions with the irrigation fluid and the pressurized gas, which, at the discretion of the attending physician or other health care professional, can be left in the heart or removed using the outer tip portion of the surgical pressurization device and the suction source of the surgical pressurization system. This procedure permits rapid testing of valvular function in a safe manner while also minimizing entry of air into the selected cardiac regions.

It is contemplated that the disclosed devices, systems, and methods can be used during any open-heart surgical procedure, including, for example and without limitation, valve repair or replacement procedures, patent foramen ovale closure procedures, and tumor or mass removal procedures. It is further contemplated that the disclosed devices, systems, and methods can be used during minimally invasive procedures, robotic procedures, and traditional full-sternotomy procedures.

In another aspect, the methods can further comprise establishing fluid communication between the suction source and the waste outlet of the surgical pressurization system. In yet another aspect, the methods can comprise activating the suction source to transport undesired blood, fluid, and gas from the heart of the subject through the opening of the outer tip portion of the delivery conduit and into the receiving chamber and the waste outlet of the surgical pressurization device.

In exemplary aspects, the step of selectively delivering at least one of the pressurized gas and the irrigation fluid can comprise activating the controller of the surgical pressurization device to selectively control delivery of the pressurized gas and the irrigation fluid to the delivery conduit. In one aspect, the methods can comprise establishing communication between the controller and the valve of the gas container. In this aspect, the methods can further comprise activating the controller to control the valve by opening and closing the valve to selectively control the flow of the pressurized gas through the outlet of the gas container. In another aspect, the methods can comprise establishing communication between the controller and the valve of the fluid container. In this aspect, the methods can further comprise activating the controller to control the valve by opening and closing the valve to selectively control the flow of the irrigation fluid through the outlet of the fluid container. It is further contemplated that the methods can comprise establishing fluid communication between the controller and the pump. It is still further contemplated that the methods can comprise activating the controller to adjust the motor of the pump to selectively control the flow of the irrigation fluid into the fluid inlet of the receiving chamber.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A surgical pressurization device for pressurizing at least one cardiac region of the subject, each cardiac region being selected from the group consisting of a left ventricle, a right ventricle, a left atrium, a right atrium, and an aorta of the subject, the surgical pressurization device comprising:
a housing defining a receiving chamber, the receiving chamber having a gas inlet configured to receive a pressurized gas and a fluid inlet configured to receive an irrigation fluid; and
a delivery conduit in fluid communication with the receiving chamber of the housing, wherein the delivery conduit has an outer tip portion defining an opening, and wherein the delivery conduit is configured to deliver both the pressurized gas and the irrigation fluid through the opening of the outer tip portion and into the selected cardiac regions of the subject.

2. The surgical pressurization device of claim 1, wherein the delivery conduit is configured to substantially simultaneously deliver both the pressurized gas and the irrigation fluid into the selected cardiac regions of the subject.

3. The surgical pressurization device of claim 1, wherein the receiving chamber of the housing further comprises a waste outlet, wherein the waste outlet is selectively connectable in fluid communication with a suction source configured to provide suction during activation, and wherein, when the waste outlet is connected in fluid communication with the activated suction source, undesired blood, fluid, and gas within the heart of the subject are selectively removed through the opening of the outer tip portion of the delivery conduit and transported into the receiving chamber, out the waste outlet of the housing, and toward the suction source.

4. The surgical pressurization device of claim 1, further comprising a controller configured to control the flow of the pressurized gas and the irrigation fluid into the delivery conduit.

5. The surgical pressurization device of claim 1, wherein the outer tip portion of the delivery conduit has a soft outer surface.

6. The surgical pressurization device of claim 1, wherein at least a portion of the housing is shaped for positioning within a hand of a user.

7. A surgical pressurization system for selectively pressurizing at least one cardiac region of the subject, each cardiac region being selected from the group consisting of a ventricle, an atrium, and the aorta of the subject, the surgical pressurization system comprising:
a gas container configured to receive a pressurized gas, the gas container having an outlet;
a fluid container configured to receive an irrigation fluid, the fluid container having an outlet; and
a surgical pressurization device comprising:
a housing defining a receiving chamber, the receiving chamber having a gas inlet in fluid communication with the outlet of the gas container and a fluid inlet in fluid communication with the outlet of the fluid container; and
a delivery conduit in fluid communication with the receiving chamber of the housing, wherein the delivery conduit has an outer tip portion defining an opening; and
a controller configured to control the flow of the pressurized gas and the irrigation fluid into the delivery conduit,
wherein the delivery conduit is configured to deliver both the pressurized gas and the irrigation fluid through the opening of the outer tip portion and into the selected cardiac regions of the subject.

8. The surgical pressurization system of claim 7, wherein the delivery conduit is configured to substantially simultaneously deliver both the pressurized gas and the irrigation fluid into the selected cardiac regions of the subject.

9. The surgical pressurization system of claim 7, further comprising a suction source configured to provide suction during activation, wherein the receiving chamber of the housing further comprises a waste outlet selectively connectable in fluid communication with the suction source, and wherein, when the waste outlet is connected in fluid communication with the activated suction source, undesired blood, fluid, and gas within the heart of the subject are selectively removed through the opening of the outer tip portion of the delivery conduit and transported into the receiving chamber, out the waste outlet of the housing, and toward the suction source.

10. The surgical pressurization system of claim 7, wherein the outer tip portion of the delivery conduit has a soft outer surface.

11. The surgical pressurization system of claim 7, wherein the gas container further comprises a valve for controlling the flow of the pressurized gas through the outlet of the gas container, and wherein the controller is connectable in communication with the valve of the gas container and configured to control the valve by opening and closing the valve to selectively control the flow of the pressurized gas through the outlet of the gas container.

12. The surgical pressurization system of claim 7, wherein the fluid container further comprises a valve for controlling the flow of the irrigation fluid through the outlet of the fluid container, and wherein the controller is connectable in communication with the valve of the fluid container and configured to control the valve by opening and closing the valve to selectively control the flow of the irrigation fluid through the outlet of the fluid container.

13. The surgical pressurization system of claim 12, further comprising a pump in communication with the outlet of the fluid container, wherein the pump has a motor configured to pump the irrigation fluid into the fluid inlet of the housing, and wherein the controller is connectable in communication with the pump and configured to adjust the motor of the pump to selectively control the flow of the irrigation fluid into the fluid inlet of the housing.

14. A method for pressurizing at least one cardiac region of a subject during a surgical procedure, each cardiac region being selected from the group consisting of a left ventricle, a right ventricle, a left atrium, a right atrium, and an aorta of the subject, the method comprising:
using a surgical pressurization device comprising:
a housing defining a receiving chamber, the receiving chamber having a gas inlet configured to receive a pressurized gas and a fluid inlet configured to receive an irrigation fluid; and
a delivery conduit in fluid communication with the receiving chamber of the housing, wherein the delivery conduit has an outer tip portion defining an opening, and wherein the delivery conduit is configured to deliver both the pressurized gas and the irrigation fluid through the opening of the outer tip portion and into the selected cardiac regions of the subject.
establishing fluid communication between an outlet of a gas container and the gas inlet of the receiving chamber, the gas container containing a pressurized gas;
establishing fluid communication between an outlet of a fluid container and the fluid inlet of the receiving chamber, the fluid container containing an irrigation fluid;
positioning the outer tip portion of the delivery conduit through a heart valve and into a first selected cardiac region of the subject; and
selectively delivering at least one of the pressurized gas and the irrigation fluid into the first selected cardiac region of the subject.

15. The method of claim 14, wherein the step of selectively delivering at least one of the pressurized gas and the irrigation fluid comprises substantially simultaneously delivering both the pressurized gas and the irrigation fluid into the first selected cardiac region of the subject.

16. The method of claim 14, wherein the heart valve of the subject is selected from the group consisting of a mitral valve, a tricuspid valve, and an aortic valve.

17. The method of claim 14, further comprising:
positioning the outer tip portion of the delivery conduit of the surgical pressurization device through a heart valve and into a second selected cardiac region of the subject; and
selectively delivering at least one of the pressurized gas and the irrigation fluid into the second selected cardiac region of the subject.

18. The method of claim 14, further comprising:
establishing fluid communication between a suction source and a waste outlet of the surgical pressurization device; and
activating the suction source to transport undesired blood, fluid, and gas from the heart of the subject through the opening of the outer tip portion of the delivery conduit and through the receiving chamber and the waste outlet of the surgical pressurization device.

19. The method of claim 14, wherein the pressurized gas comprises pressurized carbon dioxide, and wherein the irrigation fluid comprises saline solution.

20. The method of claim 14, wherein the surgical pressurization device comprises a controller positioned in operative communication with the gas container and the fluid container, the controller being configured to control the flow of the pressurized gas and the irrigation fluid into the delivery conduit, and wherein the step of selectively delivering at least one of the pressurized gas and the irrigation fluid comprises activating the controller to selectively control delivery of the pressurized gas and the irrigation fluid to the delivery conduit.

21. The method of claim 20, wherein the gas container comprises a valve for controlling the flow of the pressurized gas through the outlet of the gas container, wherein the controller is positioned in operative communication with the valve of the gas container, and wherein the step of selectively delivering at least one of the pressurized gas and the irrigation fluid comprises activating the controller to control the valve by opening and closing the valve to selectively control the flow of the pressurized gas through the outlet of the gas container.

22. The method of claim 20, wherein the fluid container comprises a valve for controlling the flow of the irrigation fluid through the outlet of the fluid container, wherein the controller is positioned in operative communication with the valve of the fluid container, and wherein the step of selectively delivering at least one of the pressurized gas and the irrigation fluid comprises activating the controller to control the valve by opening and closing the valve to selectively control the flow of the irrigation fluid through the outlet of the fluid container.

23. The method of claim 22, wherein outlet of the fluid container is in communication with a pump having a motor configured to pump the irrigation fluid into the fluid inlet of the housing, wherein the controller is in operative communication with the pump, and wherein the step of selectively delivering at least one of the pressurized gas and the irrigation fluid comprises activating the controller to adjust the motor of the pump to selectively control the flow of the irrigation fluid into the fluid inlet of the housing.
